(19) United States
(12) Patent Application Publication
(10) Pub. No.: US 2018/0098884 A1
(43) Pub. Date: Apr. 12, 2018

(54) CORNEA PROTECTOR AND SMILE EXTRA CURE UNIT INCLUDING CORNEA PROTECTOR

(71) Applicants: II Hwan KO, Seongnam-si (KR); Seong Bae PARK, Seoul (KR); Hun YANG, Yongin-si (KR); lnsik KIM, Seoul (KR)

(72) Inventors: Il Hwan KO, Seongnam-si (KR); Seong Bae PARK, Seoul (KR); Hun YANG, Yongin-si (KR); Seung Jae LEE, Seoul (KR)

(21) Appl. No.: 15/620,891

(22) Filed: Jun. 13, 2017

(30) Foreign Application Priority Data
Oct. 10, 2016 (KR) 10-2016-0130766

(51) Int. Cl. A61F 9/02; A61F 2/14; A61B 90/00

(52) U.S. Cl. CPC A61F 9/022; A61B 2090/049; A61B 90/04; A61F 2/1451

(57) ABSTRACT

Provided are a cornea protector and a SMILE extra cure unit including the cornea protector, which are used for surgery to correct a vision by generating a lenticule to be removed for vision correction while the epithelial layer is not removed by using a laser and then removing the lenticule to an incision surface having a length shorter than the diameter of the lenticule and enhance binding force of the cornea with the corrected vision by administering riboflavin and irradiating ultraviolet to a position in which the lenticule is removed.

### CROSS-REFERENCE TO RELATED APPLICATIONS

This application is based on and claims priority from Korean Patent Application No. 10-2016-0130766, filed on Oct. 10, 2016, with the Korean Intellectual Property Office, the disclosure of which is incorporated herein in its entirety by reference.

### TECHNICAL FIELD

The present disclosure relates to a small incision lenticule extraction (SMILE) extra cure unit, particularly, to a cornea protector and a SMILE extra cure unit including the cornea protector.

### BACKGROUND

In order to correct the defective vision of the eyeball, surgeries for correcting the vision by a method of changing a shape of the cornea to a desired shape by removing a part of the cornea have been frequently used, and representative surgeries are LASIK and LASEK.

The laser in-situ keratomileusis (LASIK) is a surgery method that can minimize pains and conical haze while correcting the vision and shorten a vision recovery time compared to the related art, by making a conical flap, lifting the corneal flap and setting the lifted corneal flap aside, cutting the cornea as needed by irradiating a laser to an exposed corneal parenchyma, and then covering the corneal flap set aside on the top of the cut cornea.

The laser assisted sub-epithelial keratomileusis (LASEK) is a surgery method of changing the shape of the cornea to have a desired refractive index of the cornea by making a corneal epithelial flap or removing only the corneal epithelium by using a diluted alcohol, a brush, or a laser and then cutting the cornea by irradiating the laser to the corneal parenchyma.

The LASEK surgery has advantages in that there are no complications by the corneal flap which may occur during the LASIK surgery, that is, corneal wrinkles, epithelial ingrowth, irregular flaps, and the like, the LASEK surgery is strong against physical shock, and there is less dry eye syndrome after surgery than the LASIK surgery. Further, since a thicker cornea after surgery than the LASIK surgery may remain, there is an advantage in that a frequency of a phenomenon in which the cornea is pushed or deformed, such as a keratoconus is relatively less than the LASIK surgery. However, because the epithelium is peeled off, the wound healing response is strongly induced, and as a result, corneal opacity or degeneration reaction more easily occurs than the LASIK surgery. As a result, there is a disadvantage in that the period required to use steroids for suppressing the corneal opacity or degeneration reaction is longer than that of the LASIK surgery.

The keratoconus means a progressive disease in which while a part of the cornea gradually becomes thinner, an original gentle round shape is not maintained and the cornea protrudes forward from the eyeball, and thus the vision is damaged. The cornea protrudes near the center to cause irregular astigmatism and thus, cause deterioration of the vision which is not corrected with the glasses.

The cause of the keratoconus has not yet been clearly found. Experts have supposed that inherited genetic predisposition, repetitive physical factors such as eye-rubbing habits, environmental factors that cause the physical factors, and nutrient imbalances should act synthetically. Recently, the keratoconus after the LASIK surgery or LASEK surgery occurs, and as such, the keratoconus caused after the LASIK surgery or LASEK surgery is referred to as a keratectasia. This is a phenomenon in which because a thickness of a part of the cornea is thinner than that of the periphery of the part by incising the part of the cornea with the laser, the part of the cornea does not stand the pressure in the eye to be pushed.

FIG. 1 is a photograph of comparing a normal cornea and a keratoconus.

FIG. 1A illustrates cross sections of the normal cornea and the keratoconus, FIG. 1B illustrates corneal topographies of the normal cornea and the keratoconus, and FIG. 1C illustrates thicknesses for each part of the cornea. Referring to FIG. 1C, it can be seen that the thickness of the keratoconus is smaller than that of the normal cornea, and the thickness of the cornea is not constant.

Various methods for treating the keratoconus have been proposed and used, but basically, it is required to minimally suppress occurrence of the keratoconus. Particularly, even in a vision correction surgery of removing the predetermined part of the cornea by using the laser, a surgery apparatus (system) and a surgery method for preventing the keratoconus that may occur after surgery have been required.

### SUMMARY

The present disclosure has been made in an effort to provide a cornea protector having advantages of uniformly maintaining a wet state of the surface of the cornea, serving as a secretion barrier to prevent various secretions secreted from the eyelid or conjunctiva from being invaded to the surface of the cornea of a part to be irradiated with ultraviolet light by mixing the tears, and performing a function of a mask that protects the cornea, the conjunctiva and the sclera tissue other than portions where ultraviolet irradiation is required from ultraviolet light.

Further, the present disclosure has been made in an effort to provide a SMILE extra cure unit including a cornea protector having advantages of changing a shape of the cornea to have a desired refractive index by generating a lenticule to be removed while the epithelial layer is not removed by using a laser and then separating and removing the lenticule through an incision surface having a length smaller than a diameter of the lenticule by using a separating tool and maximizing a vision correction effect and a cornea enhancement effect by using the cornea protector when binding force of the cornea after vision correction is enhanced by administering riboflavin and irradiating ultraviolet light to a position where the lenticule is removed.

An exemplary embodiment of the present disclosure provides a cornea protector which is made of a material absorbing water well and having a property of interfering with transmission of ultraviolet light and has a ring shape with a predetermined thickness, in which a size of an empty space therein may be selected for various sizes to correspond to the size of a ultraviolet light irradiation region to be used to a patient with corneal surgery and an inner curvature of the empty space therein corresponds to the curvatures of the cornea and the eyeball.

Another exemplary embodiment of the present disclosure provides a SMILE extra cure unit including a curved contact glass, a curved contact glass controller, a laser, a riboflavin administering unit, a cornea protector, and an ultraviolet light irradiator. The curved contact glass is prepared according to a size of the cornea. The curved contact glass controller controls a distance between the curved contact glass and the cornea and fixes the curved contact glass to the cornea or separates the curved contact glass from the cornea by using pressure. The laser incises a predetermined portion of the cornea by adjusting energy of irradiated light. The riboflavin administering unit administers riboflavin to a site of the cornea which is incised and then removed by the laser. The cornea protector is disposed on the periphery tissue of the cornea administered with the riboflavin. The ultraviolet light irradiator irradiates ultraviolet light to the cornea after the riboflavin is cleaned. Herein, the cornea protector includes a plurality of cornea protectors having various sizes of the empty space therein.

According to the cornea protector and the SMILE extra cure unit including the cornea protector according to the present disclosure, it is possible to efficiently correct a vision by accurately generating and removing a lenticule by using a curved contact glass and a curved contact glass controller which controls a distance between the curved contact glass and the cornea and fixes the curved contact glass to the cornea or separates the curved contact glass from the cornea by using pressure and maximally increase the strength of a treated corneal portion by administering riboflavin to a site where the lenticule is removed and then irradiating ultraviolet light having constant energy.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A, 1B and 1C are photographs of comparing a normal cornea and a keratoconus.

FIG. 2 illustrates a configuration of a SMILE extra cure unit including a cornea protector according to the present disclosure.

FIGS. 3A and 3B illustrate a cornea protector provided in the present disclosure.

FIG. 4 illustrates a SMILE extra vision correction method according to the present disclosure.

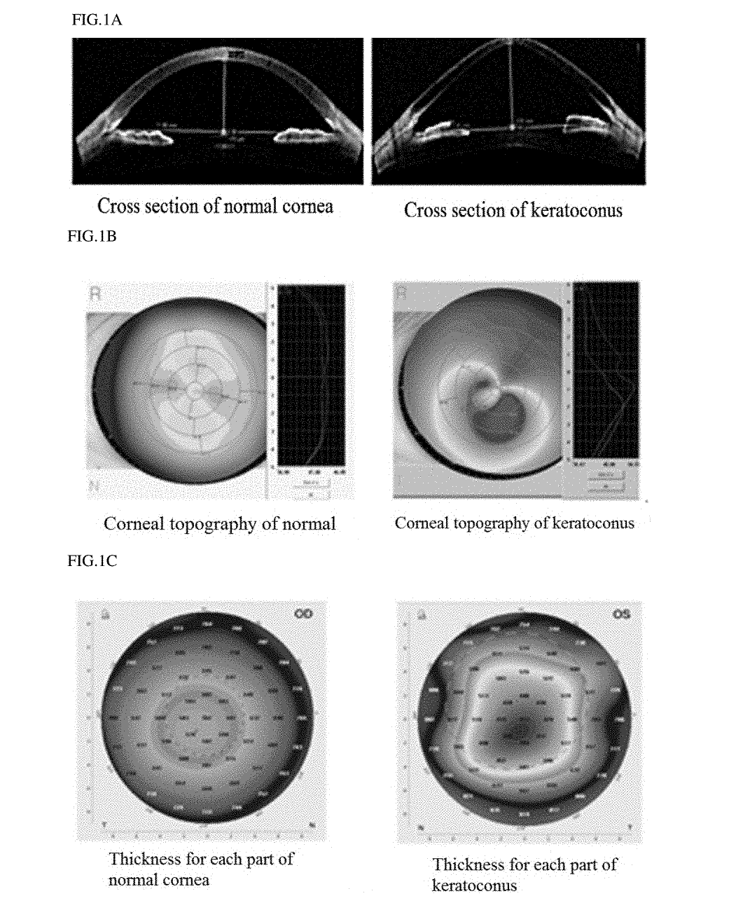

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

A SMILE extra cure unit according to an embodiment of the present disclosure is used for correcting a vision and enhancing the strength of the cornea with the corrected vision. The correction of the vision is achieved by generating a lenticule that needs to be removed without removing the epithelial layer by using a laser and then separating and removing the lenticule from the cornea as an incision surface with a length shorter than a diameter of the lenticule. The strength of the cornea may be enhanced by maximally increasing the strength of the cornea at the cured portion by irradiating ultraviolet light having constant energy after administering riboflavin into a site from which the lenticule is removed. When the strength of the cornea is enhanced, the cornea protector according to the embodiment of the present disclosure is used to absorb excessive moisture around the cornea, prevent impurities from being dispersed to the surface of the cornea, and irradiate ultraviolet light to only a predetermined portion requiring treatment when the ultraviolet light is irradiated, thereby improving safety of the surgery by preventing the ultraviolet light from being irradiated to portions such as the periphery of the cornea, the conjunctiva, and the sclera in which the irradiation of the ultraviolet light is not required.

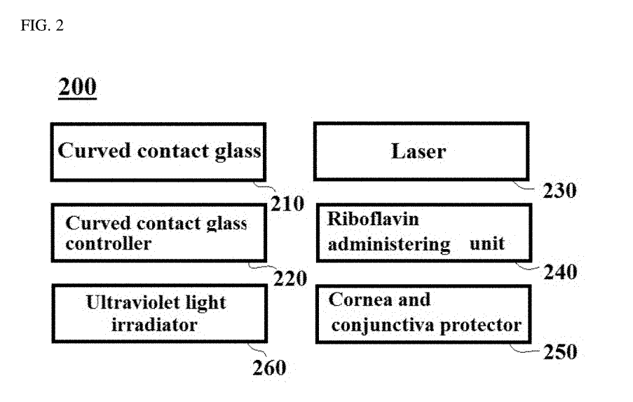

FIG. 2 illustrates a configuration of a SMILE extra cure unit including a cornea protector according to the present disclosure.

Referring to FIG. 2, a SMILE extra cure unit 200 including the cornea protector includes a curved contact glass 210, a curved contact glass controller 220, a laser 230, a riboflavin administrating unit 240, a cornea protector 250, and a ultraviolet irradiator 260.

The curved contact glass 210 is made of a transparent plastic material with a size of the cornea. The curved contact glass 210 serves to prevent the movement of the cornea by contacting the cornea. The curved contact glass controller 220 may prevent the movement of the cornea by attaching the curved contact glass 210 to the cornea and then applying predetermined pressure or separate the curved contact glass 210 from the cornea by removing the applied pressure for attachment.

The laser 230 is used for incising the cornea of the predetermined portion by adjusting irradiated light energy and controlling a depth of the irradiated light. Types of used laser 230 are various and used examples are various according to a position and a thickness of the incised cornea, but even in the present disclosure, since the laser known in the related art is used, the detailed description of the laser will be omitted. The riboflavin administrating unit 240 is used for improving the tissue strength of the cornea in which the treatment is completed while the keratoconus is treated.

The cornea protector 250 may be made of an open-cell foam synthetic polymer having properties of facilitating insertion and ejection compared to Vaseline gauze which has been used in the related art, decreasing a foreign action, absorbing water well, and interfering with the transmission of the ultraviolet light. The open-cell foam synthetic polymer may be implemented by Merocel® of Medtronic Corporation. In the present disclosure, the cornea protector 250 capable of performing both of two functions to be described below is proposed.

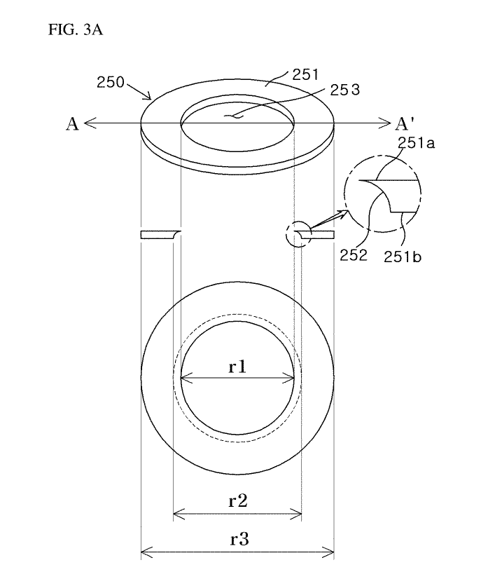

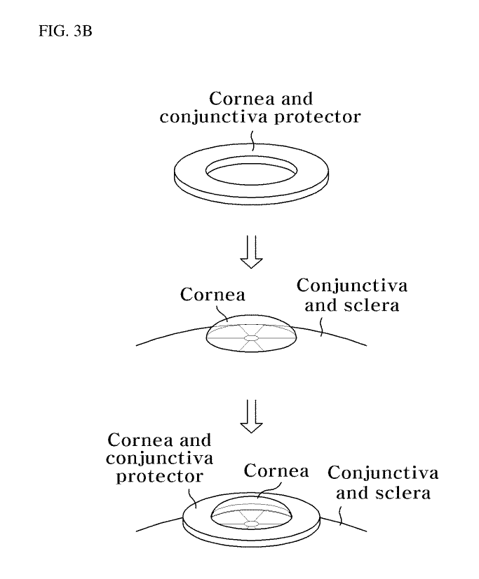

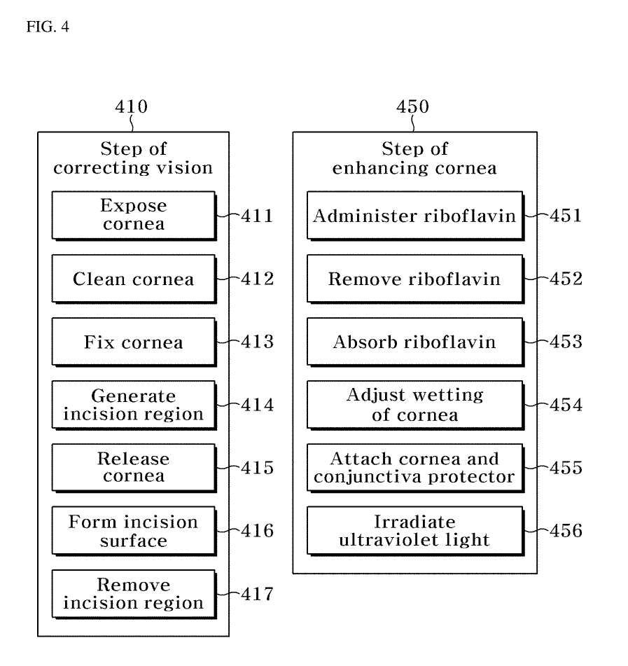

FIG. 3 illustrates the cornea protector according to the exemplary embodiment of the present disclosure. FIG. 3A illustrates a perspective view, a cross-sectional view, and a plan view of the cornea protector and FIG. 3B illustrates a used example of the cornea protector.

As illustrated in FIG. 3A, the cornea protector 250 is constituted by a ring-shaped annular body 251 of which an aperture 253 is formed at the center. The cornea is inserted to the aperture 253. The annular body 251 is formed with a predetermined thickness. Preferably, the annular body 251 may be formed with a thickness of 1 mm to 4 mm. An upper surface 251a and a lower surface 251b contacting the eyeball of the annular body 251 are formed with different inner diameters. That is, an inner diameter r1 of the upper surface 251a is formed to be smaller than an inner diameter r2 of the lower surface 251b. A curved connection portion 252 connecting the inner periphery of the upper surface 251a and the inner periphery of the lower surface 251b is formed by a curved surface having a predetermined curvature. Preferably, the curved connection portion 252 may be formed with a curvature of 7.8 to 9.2 mm. The cornea protector 250 may be stably seated on the curved surface of the eyeball including the cornea and the conjunctiva by forming the curved connection portion 252 with the curvature.

Further, since the size of the cornea varies according to the human, an outer diameter r3 of the annular body 251, the inner diameter r1 of the upper surface 251a, and the inner diameter r2 of the lower surface 251b in the cornea protector 250 need to vary according to the size of the cornea. In the present disclosure, considering these sizes, a plurality of cornea protectors 250 with the outer diameter r3 of 1 mm unit and the inner diameter r1 of the upper surface 251a and the inner diameter r2 of the lower surface 251b of 0.1 mm units is provided and selectively used according to a size of the cornea.

For example, when the outer diameter r3 has a range of 15 to 20 mm, the cornea protectors having r3 of 15 mm, 16 mm, 17 mm, 18 mm, 19 mm, and 20 mm, respectively, may be provided. Further, when the inner diameter r1 of the upper surface 251a has a range of 6 to 8 mm, the cornea protectors having r1 of 6.0 mm, 6.1 mm, . . . , 7.8 mm, 7.9 mm and 8.0 mm, respectively, may be provided.

Two important functions of the cornea protector 250 will be described below.

The ultraviolet light irradiator 260 is a device of irradiating ultraviolet light having a predetermined intensity to the top of the cornea when a predetermined time elapses after riboflavin is administered to the cornea. The degree of irradiating the ultraviolet light is generally expressed by energy, and in the present disclosure, energy of 0.3 to 2.7 joules may be transmitted to the cornea. In order to achieve this, for example, ultraviolet light of 30 milliwatts (mW) needs to be irradiated to the cornea for 90 seconds. An irradiation time of the ultraviolet light may be changed to 10 to 90 seconds according to an energy amount to be irradiated. Of course, the energy may be transmitted by setting power of the ultraviolet light to power having a size other than 30 mW and changing the irradiation time corresponding thereto.

The SMILE extra cure unit 200 according to the present disclosure improves a conventional SMILE curing method. The conventional SMILE (Small Incision Lenticule Extraction) curing method is a surgery method of changing a curvature of the cornea by incising the corneal parenchyma in a desired shape by using laser equipment and then separating and removing the incised corneal parenchyma. The conventional SMILE curing method has an effect in vision correction, but does not efficiently prevent a keratoconus phenomenon from being generated in a weakened cornea after correction of the vision.

In the present disclosure, a method of enhancing the cornea is added to the conventional SMILE curing method, and when the method of enhancing the cornea is additively performed, the corneal surface in a wet state is maintained in a clear state to uniformly perform the ultraviolet light irradiation and maximally protect the peripheral cornea, the conjunctiva, and the sclera (see FIG. 3B) when the ultraviolet light is irradiated, and thus a name of the SMILE extra in the title of the invention is included.

Hereinafter, the SMILE extra vision correction method of correcting the vision by using the SMILE extra cure unit 200 including the cornea protector will be described.

FIG. 4 illustrates a SMILE extra vision correction method according to the present disclosure.

Referring to FIG. 4, a SMILE extra vision correction method 400 may be largely divided to steps of correcting the vision (410) and enhancing the cornea (450).

The lenticule used in the following description has a similar shape to the shape of the cornea incised in LASIK surgery or LASEK surgery for correcting the vision. The LASIK surgery or the LASEK surgery has a difference in a method of changing the shape of the cornea, by making the cornea having the same shape as the shape of the portion burned and removed in the SMILE extra surgery, that is, the lenticule and separating and removing the lenticule from the cornea if this form of cornea is incised by an excimer laser, that is, burned and removed by a laser.

In the step of correcting the vision (410), a predetermined portion (the lenticule) of the cornea is removed for correcting the vision, and to this end, steps of exposing the cornea (411), cleaning the cornea (412), fixing the cornea (413), generating an incision region (414), releasing the cornea (415), forming an incision surface (416) and removing the incision region (417) are performed.

In the step of exposing the cornea (411), the cornea is exposed to the outside by opening eyelids by using an eyelid speculum (not illustrated). In the step of cleaning the cornea (412), the exposed cornea is cleaned and a predetermined amount of water remains in the cleaned cornea.

In the step of fixing the cornea (413), the curved contact glass 210 moves to the cornea and contacts the cornea, and then the curved contact glass 210 is in close contact with the cornea by applying constant suction pressure to the curved contact glass 210 to prevent the cornea from moving during treatment. In this case, in order to prevent the patient's eyes from moving, the patient needs to continuously look at the fixed light source. In the step of generating the incision region (414), the cornea with a predetermined depth is incised with a predetermined size by using the laser 230. In this case, the incised portion is referred to as the lenticule.

In the step of releasing the cornea (415) performed after generating the lenticule by using the laser 230, the curved contact glass 210 may be easily separated from the cornea by removing the pressure applied to the curved contact glass 210 in the step of fixing the cornea (413).

In the step of forming the incision surface (416), when the lenticule which is a corneal flap incised by using a tool such as a forceps is removed, the incision surface becomes a passage through which the forceps and the lenticule move, and a long incision surface through which the lenticule may pass without modification is not generated, but an incision surface having a minimal length to be removed even though the lenticule is modified is formed, thereby minimizing a recovery time of the incision surface after removing the lenticule.

In the step of removing the incision region (417), the forceps is inserted between the incision surfaces generated in the step of forming the incision surface (416) to extract the lenticule which is the incised conical flap.

In the step of correcting the vision (410), the epithelial layer covering the cornea to generate the lenticule is not removed and a conical flap structure is not generated. In a conventional LASEK surgery, in order to incise the parenchyma of the cornea, the epithelial layer needs to be removed to strongly cause the resulting inflammation and immune response, and thus, regression of myopia or astigmatism due to corneal opacity or regeneration after surgery relatively frequently occurs. In order to maximally suppress the regression, mitomycin C as one of anticancer agents is diluted and applied to the surgical site or the use of steroid eye drops for a long time after surgery is inevitable.

As described above, in the present disclosure, the lenticule is removed without removing the epithelial layer, and accordingly, steroids can be used less or shorter than when the epithelial layer is removed and thus, the risk of developing glaucoma is reduced. The epithelial layer has a thickness of 50 micrometers (μm), and the fact that the epithelial layer is not removed means that the cornea becomes thicker as much, and an advantage of the feature exhibits an effect (to be described below) in the subsequent step of enhancing the cornea (450).

Further, in the conventional LASIK surgery, since a necessary corneal flap structure is not generated, the consumption of the cornea is reduced and thus, it is easy to expect that stability of the cornea will be improved.

In the present disclosure, a refractive index of the cornea is changed by removing the lenticule to correct the vision and the step of enhancing the cornea (450) to be described below is performed, and thus, it is proposed that the tissue of the thinned cornea is solidified in the step of correcting the vision (410).

In the step of enhancing the cornea (450), riboflavin is administered to the region where the lenticule is removed in the step of correcting the vision (410) to enhance the corneal tissue in the region where the lenticule is removed by irradiating the ultraviolet light. In order to perform the function, steps of administering riboflavin (451), removing the riboflavin (452), absorbing the riboflavin (453), adjusting wetting of the cornea (454), attaching the cornea protector (455) and irradiating ultraviolet light (456) are performed.

In the step of administering the riboflavin (451), a riboflavin (vitamin B2) solution is administered to the region where the lenticule is removed by using the riboflavin administering unit 240.

In the step of removing the riboflavin (452), after performing the step of administering the riboflavin (451), the remaining riboflavin flowing out through an incision slit is cleaned and removed with physiological saline. In the step of absorbing the riboflavin (453), the riboflavin sufficiently penetrates into the cornea for about 30 seconds to 3 minutes. In the step of adjusting the wetting of the cornea (454), a thin and clear water layer is formed on the surface of the cornea by cleaning the remaining eyelid and conjunctival secretions by using physiological saline.

In the step of attaching the cornea protector (455), the cornea protector 250 is placed on the surface of the cornea. In this case, the cornea protector 250 is positioned to accurately expose only a corneal portion to be irradiated with ultraviolet light and protect the peripheral cornea, the conjunctiva, and the sclera without requiring irradiation of the ultraviolet light. When various types of cornea protectors 250 are selected according to a size of the cornea of the patient, it is not difficult to expose only the corneal portion to be irradiated with the ultraviolet light. The cornea protector 250 placed on the cornea prevents secretions secreted from the eyelid or the conjunctiva from flowing into the surface of the cornea, and the surface of the cornea is maintained in a clear state.

In the step of irradiating the ultraviolet light (456), the ultraviolet light having constant energy is irradiated to the cornea administered with the riboflavin and strong binding between collagen fibers in the cornea is formed by the reaction of the ultraviolet light and the riboflavin solution. In this case, the cornea protector 250 is irradiated with the ultraviolet light as it stands. As a result, since the region irradiated with the ultraviolet light may be precisely masked, the ultraviolet light may be minimally irradiated to the cornea to which the riboflavin is not administered and a clear surface of the cornea is maintained and thus uniform irradiation of the ultraviolet light is possible.

The cornea has a structure in which a plurality of collagen layers with predetermined thicknesses is stacked and serves as a convex lens that keeps transparency and collects light. The cornea needs to maintain a shape which is not influenced by waterproofing of the eye and pressure of a vitreous body due to constant rigidity, but in a patient with the keratoconus, the cornea is easily modified and protrudes because the cornea dose not overcome the pressure inside the eyeball due to weakened rigidity of the cornea.

When the cornea is soaked with the riboflavin and then irradiated with the ultraviolet light with constant energy, new binding between the collagen layers configuring the cornea is made and thus the framework of the cornea becomes strong.

As described above, in the present disclosure, the riboflavin may be administered without removing the epithelial layer having a thickness of about 50 μm and thus there is another additional advantage.

In the case of performing the step of irradiating the ultraviolet light (456) to the cornea while the epithelial layer is removed, since the ultraviolet light is irradiated to the cornea thinned by the thickness of the epithelial layer, there is a problem in that the ultraviolet light passes through the core of the cornea, for example, may have a negative effect on a deeper structure of the eye such as lenses and retinas. In the present disclosure, the epithelial layer of the cornea may be irradiated with the ultraviolet light in a preserved state to prevent the ultraviolet light from penetrating to an unnecessary depth.

Referring to FIG. 3 and the step of irradiating the ultraviolet light (456), it can be seen that the cornea protector 250 proposed in the present disclosure protects the peripheral corneal tissue, the conjunctiva, and the sclera, which don not require the corneal enhancement by uniformly irradiating the ultraviolet light by maintaining the surface of the cornea in a clear state when irradiating the ultraviolet light and irradiating the ultraviolet light to only the desired portion.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A cornea protector which is installed on the cornea before irradiating ultraviolet light and removed after irradiating ultraviolet light, the cornea protector comprising:
a ring-shaped annular body which is made of an open-cell foam synthetic polymer absorbing water and interfering with transmission of the ultraviolet light and has an aperture at the center,
wherein the annular body has a predetermined thickness, an inner diameter of an upper surface is smaller than an inner diameter of a lower surface contacting the eyeball, a curved connection portion connecting an inner periphery of the upper surface and an inner periphery of the lower surface has a predetermined curvature, the curvature is set to a size corresponding to curvatures of the cornea and the eyeball, and the size of the aperture is formed with a size corresponding to a size of an ultraviolet irradiation region to be used to a patient with corneal surgery.

2. The cornea protector of claim 1, wherein the thickness is any one selected from 1 to 4 mm and the curvature is any one selected from 7.8 to 9.2 mm.

3. A SMILE extra cure unit comprising:
a curved contact glass prepared with a size corresponding to a size of the cornea;
a curved contact glass controller which controls a distance between the curved contact glass and the cornea and fixes the curved contact glass to the cornea or separates the curved contact glass from the cornea by using pressure;
a laser which incises a predetermined portion of the cornea by adjusting energy of irradiated light;
a riboflavin administering unit which administers riboflavin to a site of the cornea which is incised and then removed by the laser;
the cornea protector of claim 1 which is disposed on the cornea administered with the riboflavin; and
an ultraviolet light irradiator which irradiates ultraviolet light to the cornea after the riboflavin is cleaned.
